(19) United States
(12) Patent Application Publication
(10) Pub. No.: US 2008/0057062 A1
(43) Pub. Date: Mar. 6, 2008

(54) Agent for Inducing senescence and apoptosis of cancer cell

(75) Inventors: Renu WADHWA, Ibaraki (JP); Sunil KAUL, Ibaraki (JP)

Correspondence Address: GREENBLUM & BERNSTEIN, P.L.C., 1950 ROLAND CLARKE PLACE, RESTON, VA, 20191, US

(73) Assignee: NATIONAL INSTITUTE OF ADVANCED INDUSTRIAL SCIENCE AND TECHNOLOGY, Tokyo (JP)

(21) Appl. No.: 11/778,763

(22) Filed: Jul. 17, 2007

(30) Foreign Application Priority Data
Jul. 18, 2006 (JP) 2006-196050

(51) Int. Cl. A61K 31/395; A61K 31/715; A61P 43/00

(52) U.S. Cl. 424/138.1; 514/44

(57) ABSTRACT

An object of the present invention is to further examine the functions of the above CARF in detail so as to develop a novel drug through elucidation of such functions. The present invention provides an anticancer agent which comprises an agent for suppressing CARF expression or an agent for inactivating CARF as an active ingredient.

### TECHNICAL FIELD

The present invention relates to an agent for inducing senescence and apoptosis of cancer cell using an agent for suppressing CARF expression or an agent for inactivating CARF.

### BACKGROUND ART

The INK4a gene locus located on chromosome 9p21 is a site at which mutation occurs frequently in human cancer cells. The gene locus encodes 2 protein factors, p161^{NK4a} and ARF (alternative reading frame protein), which are completely distinct structurally from each other. Within the last decade, a large amount of evidence concerning cell senescence, cell division control, and cancer-derived cells has been accumulated, thus supporting the fact that these 2 protein factors are major cancer suppressors (see Jacobs, J. J., Kieboom, K., Marino, S., DePinho, R. A., and van Lohuizen, M. (1999) Nature 397, 164-168; Vogt, M., Haggblom, C., Yeargin, J., Christiansen-Weber, T., and Haas, M. (1998) Cell Growth Differ 9, 139-146; and Wei, W., Hemmer, R. M., and Sedivy, J. M. (2001) Mol Cell Biol 21, 6748-6757). It has been reported that the functional deficiencies of these cancer suppressors are induced by different mechanisms including gene deficiencies, mutations, silencing mediated by methylation, and the like in various cancers. It has also been reported that cell division suppression due to p16^{INK4a} is caused by the suppression of pRB phosphorylation (see Serrano, M., Lee, H., Chin, L., Cordon-Cardo, C., Beach, D., and DePinho, R. A. (1996) Cell 85, 27-37). In the meantime, cell division suppression due to ARF promotes p53 functions by inhibiting HDM2 (human double minute-2 oncoprotein), which is a p53 antagonist (see Weber, J. D., Jeffers, J. R., Rehg, J. E., Randle, D. H., Lozano, G., Roussel, M. F., Sherr, C. J., and Zambetti, G. P. (2000) Genes Dev 14, 2358-2365; Lloyd, A. C. (2000) Nat Cell Biol 2, E48-50; and Zhang, Y., Xiong, Y., and Yarbrough, W. G. (1998) Cell 92, 725-734). Control of a cancer suppression pathway mediated by p53 and Rb is the central aspect of cell senescence and canceration. Findings and molecular analysis concerning the regulators thereof are very important.

To understand the control of the ARF-p53 pathway, we have searched for a partner factor that binds to p19^{ARF} (mouse ARF) using a yeast interactive screen and then isolated a novel protein CARF (collaborator of ARF). We have already clarified the following facts concerning CARF (Wadhwa, R., Sugihara, T., Hasan, M. K., Duncan, E. L., Taira, K., and Kaul, S. C. (2003) Exp Gerontol 38, 245-252; Hasan, M. K., Yaguchi, T., Sugihara, T., Kumar, P. K., Taira, K., Reddel, R. R., Kaul, S. C., and Wadhwa, R. (2002) J Biol Chem 277, 37765-37770; and Hasan, M. K., Yaguchi, T., Minoda, Y., Hirano, T., Taira, K., Wadhwa, R., and Kaul, S. C. (2004) Biochem J 380, 605-610).

(i) CARF is a nuclear protein richly containing serine.

(ii) CARF is located on human chromosome 4p35 and mouse chromosome 8.

(iii) Human CARF and mouse CARF share homology of 84.2%.

(iv) CARF binds to human ARF and mouse ARF.

(v) ARF-CARF complex is localized in peripheral region.

(vi) ARF-CARF complex activates p53 functions.

(vii) CARF can bind to p53 in the absence of ARF, so as to prevent denaturation due to HDM2.

### DISCLOSURE OF THE INVENTION

An object of the present invention is to further examine the functions of the above CARF in detail so as to develop a novel drug through elucidation of such functions.

As a result of intensive studies, the present inventors have obtained the following new findings concerning CARF functions, which suggest that CARF is an important control factor in the ARF-p53-HDM2 pathway and that CARF plays an important role in cell division and DNA damage response.

(i) CARF binds to HDM2.

(ii) CARF is denatured by the HDM2-dependent proteasome pathway.

(iii) CARF behaves as an HDM2 transcriptional suppressor.

(iv) CARF expression levels are increased in senescent cells. Furthermore, when CARF is expressed in large amounts, normal human cells will be pre-mature senescent cells.

(v) CARF expression reaches its peak in the G2 phase. CARF is involved in cell cycle regulation.

(vi) CARF plays a central role in DNA damage response of cell.

(vii) CARF overexpression in transformed human cells induces the G2 growth arrest and siRNA-mediated silencing promotes apoptosis.

Based on these findings, the present inventors have further examined the effects of CARF on cancer cells. As a result, the present inventors have obtained a completely surprising finding that inhibition of CARF induces senescence and apoptosis of cancer cell, and thus they have completed the present invention.

Specifically, the present invention is as follows.

(1) An anticancer agent which comprises an agent for suppressing CARF expression or an agent for inactivating CARF as an active ingredient.

(2) The anticancer agent according to (1) above, wherein the anticancer agent is an agent for inducing senescence or apoptosis of cancer cell.

(3) The anticancer agent according to (1) above, wherein the agent for suppressing CARF expression is siRNA or siRNA expression vector to target CARF.

(4) The anticancer agent according to (3) above, wherein the siRNA is a double-stranded oligonucleotide consisting of the sense strand represented by SEQ ID NO: 5 and the antisense strand represented by SEQ ID NO: 6.

(5) The anticancer agent according to (1) above, wherein the agent for inactivating CARF is an antibody against CARF.

(6) The anticancer agent according to (5) above, wherein the antibody against CARF is a monoclonal antibody.

### BRIEF DESCRIPTION OF THE DRAWING

FIG. 1 shows photographs of Western blotting and immunostaining showing that the above siRNAs of the present invention specifically suppress CARF expression.

FIG. 2 shows photographs of TUNEL staining showing that the siRNAs of the present invention induce apoptosis of Hela cells.

FIG. 3 shows photographs showing the results of analyzing the CARF protein obtained in Example 2, 2) by CBB staining and Western blotting.

FIG. 4 is a flowchart showing the outline of the method for preparing an anti-CARF antibody of the present invention.

FIG. 5 is a photograph showing the results of testing the specificity of the polyclonal antibody and the monoclonal antibody of the present invention to CARF by Western blotting and immunoprecipitation analysis.

FIG. 6 shows the results of immunostaining of CARF using the polyclonal antibody and the monoclonal antibody of the present invention.

### PREFERRED EMBODIMENTS OF THE INVENTION

The nucleotide sequence of the full-length gene of the above CARF is represented by SEQ ID NO: 1, and the amino acid sequence of the CARF protein is represented by SEQ ID NO: 2.

According to the present invention, senescence or apoptosis of cancer cell is induced by using a drug that inhibits the effects of CARF, so as to cause exertion of an anti-cancer effect.

An example of such drug that inhibits the effects of CARF is an agent for suppressing CARF expression or an agent for inactivating CARF. Specific examples of such agent for suppressing CARF expression include a CARF-targeting siRNA, ribozyme, and antisense oligonucleotide.

Such siRNA to target CARF is an oligonucleotide which basically comprises double-stranded RNAs complementary to each other. One (sense strand) of the siRNA contains a region comprising any sequence (19 nucleotides, for example) in the nucleotide sequence of mRNA corresponding to a structural gene portion of a target CARF gene (SEQ ID NO: 1). Upon selection of the region, a region having a GC content ranging from 30% to 55% is selected, for example. Moreover, the other (antisense strand) of the siRNA has a nucleotide sequence complementary to the above sense strand. The sense or antisense strand may have a 0- to 3-nucleotide addition sequence on its 3′ end. In addition, siRNA used in examples described later was designed based on a nucleotide sequence ranging from nucleotide 168 to nucleotide 286 of the CARF gene, but the present invention is not particularly limited thereto.

The above designed sense and antisense RNAs can separately be chemically synthesized according to conventional methods, for example. The thus synthesized RNAs are annealed to form a double strand by heating in a solution, so as to prepare siRNA. Such siRNA can be introduced into living bodies using liposome, for example.

Another method involves synthesizing DNAs corresponding to the above sense RNA and antisense RNA, respectively, linking the DNAs to form a sense strand DNA-linker-antisense strand DNA, and then introducing the thus linked DNAs into an expression vector, so as to be able to prepare an anticancer agent. With the use of the thus obtained recombinant vector, the incorporated DNAs are transcribed within cells, a low-molecular-weight double strand “hairpin” RNA (shRNA) is expressed, and then the RNA is cleaved by an intracellular enzyme, so that siRNA is produced.

Examples of vectors to be used herein include nonviral vectors, adenovirus vectors, and lentivirus vectors.

Meanwhile, the above sense strand DNA and antisense strand DNA are introduced into different expression vectors, separately expressed within cells, and then annealed to each other within the cells. In this manner, siRNA may also be generated. The use of these expression vectors enables continuous production of siRNA in vivo or within cells and thus is more preferable.

An antisense oligonucleotide to be used in the present invention is an RNA molecule that suppresses the expression of mRNA which is produced based on the expression of a CARF gene within cells. The RNA molecule has a nucleotide sequence with a nucleotide length between 15 and 30, which is complementary to a nucleotide sequence in mRNA corresponding to CARF. As the structure of such antisense oligonucleotide, that of a natural phosphodiester type oligomer can also be used. To avoid cleavage by nuclease, phosphorothioate type, phosphorodithioate type, phosphoroamidate type, methylphosphonate type, or methylphosphonothioate type oligomer may also be used. Furthermore, a base portion or a ribose portion may also be chemically modified. These antisense oligonucleotides are designed and produced in accordance with conventional methods.

Furthermore, a ribozyme to be used in the present invention is an RNA molecule that recognizes mRNA which is produced based on the expression of a CARF gene within cells and then cleaves the mRNA. The RNA molecule has a nucleotide sequence region complementary to a nucleotide sequence in mRNA corresponding to CARF and a loop structure region. Such ribozyme is designed based on such nucleotide sequence in the mRNA corresponding to CARF and then synthesized by a conventional method.

Moreover, when the above natural antisense oligonucleotide or ribozyme is used for cancer treatment, it is preferable to insert DNA encoding such oligonucleotide or ribozyme into the above vector for gene therapy, introduce the vector into a living body, and then cause continuous expression of the above antisense RNA or ribozyme in vivo.

Furthermore, an example of an agent for inactivating CARF, which is used in the present invention, is an antibody against CARF. Such antibody may be either a polyclonal antibody or a monoclonal antibody.

A polyclonal antibody is obtained by immunizing an animal such as a mouse, a rabbit, a rat, or a goat with CARF, and collecting the serum from the animal, preferably followed by further isolation and purification. Isolation and purification can be carried out by adequately combining conventional protein isolation and purification means including centrifugation, dialysis, salting out, DEAE-cellulose, and chromatography using protein A agarose and the like.

Furthermore, a monoclonal antibody can be obtained by a hybridoma method. Specifically, after immunization of the above animal such as a mouse with CARF, spleen cells are collected, the cells are fused to myeloma cells, the thus obtained fusion cells are subjected to screening, and then cloning is further repeated, so that cells producing a monoclonal antibody against CARF are obtained. Through culture of the cells in medium, a monoclonal antibody against CARF can be obtained.

For production of the above polyclonal antibody and monoclonal antibody, an adjuvant such as Freund's adjuvant can also be used upon immunization of animals.

### EXAMPLES

Examples of the present invention are hereafter described, although the present invention is not limited thereto.

### Example 1

1) Preparation of CARF siRNA

21-nucleotide siRNAs (corresponding to a nucleotide sequence ranging from nucleotide 168 to nucleotide 286 in the gene of SEQ ID NO: 1) to target CARF were chemically synthesized using phosphoroamidite. The synthesized RNAs were deprotected and then purified via gel extraction. The sequences of siRNAs used as controls and the sequences of CARF-targeting siRNAs are as shown below.

| | |
|---|---|
| Control siRNA | |
| 5′-AAGACCGAGUCCAUGAGGCUT-3′ | (SEQ ID NO:3) |
| 5′-GCCUCAUGGACUCGGUCUUUT-3′ | (SEQ ID NO:4) |
| CARF-targeting siRNA | |
| 5′-CGGAGUACCUGAGCCAGAAUT-3′ | (SEQ ID NO:5) |
| 5′-UUCUGGCUCAGGUACUCCGUT-3′ | (SEQ ID NO:6) |

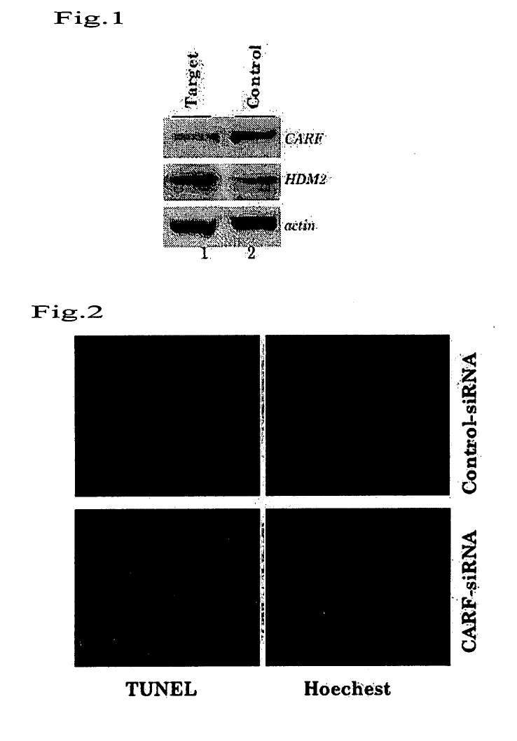

For annealing of siRNAs, each RNA strand (20 μM) was heated at 90° C. for 1 minute in an annealing buffer (100 mM potassium acetate, 30 mM HEPES-KOH at pH 7.4, and 2 mM magnesium acetate) and then cooled to 37° C. for 1 hour. Transfection of double-stranded siRNA was carried out using an oligofectamin reagent (Invitrogen Corporation). Cells in 12 wells were transfected using 1 to 5 μl of 20 μM siRNA. 24 to 48 hours after transfection, experiments were conducted by Western blotting and immunostaining using an anti-CARF antibody. FIG. 1 shows the result. As is clear from the result, the above siRNAs specifically suppressed CARF expression.

2) Induction of Apoptosis of Cancer Cells by CARF siRNA

TUNEL staining was performed for each culture well in order to detect apoptosis. This method is used to detect cleavage in a genomic DNA strand accompanying apoptosis through fluorescence labeling of the 3′-OH group after cleavage.

The CARF-specific double-stranded siRNAs and the control double-stranded siRNAs having no effect on CARF expression obtained in 1) above were separately introduced into Hela cells by a method similar to that in 1) above. After siRNA transfection, cells in each CARF culture well used therein were subjected to TUNEL staining using a DeadEnd™ Fluorometric TUNEL System (Promega Corporation). Apoptosis was then detected. As shown in FIG. 2, cells that had undergone apoptosis because of CARF expression suppressed by the CARF-specific siRNA emitted green fluorescence. In the case of the control siRNAs, no changes were observed in CARF expression, and cells undergoing apoptosis were not observed. Cell nuclei were stained using a DNA-binding reagent (Hoechest), and blue cell nuclei were observed.

### Example 2

PCR was performed using cDNA derived from human testicular cells and the following primers.

| | |
|---|---|
| 5′-GGATCCATGGCGCAGGAGGTG-3′ | (SEQ ID NO:7) |
| 5′-GTCGACTAGTAATTCTTGAGGA-3′ | (SEQ ID NO:8) |

After subcloning, the DNA region amplified by PCR was cleaved with BamH I and Sal I. The DNA fragment after cleavage was cloned into an Escherichia coli expression vector pQE30 (Qiagen) using Sal I and BamH I sites. A His tag had been incorporated in the pQE30 vector. The cDNA sequence was confirmed by sequencing.
2) Expression of Human CARF Protein

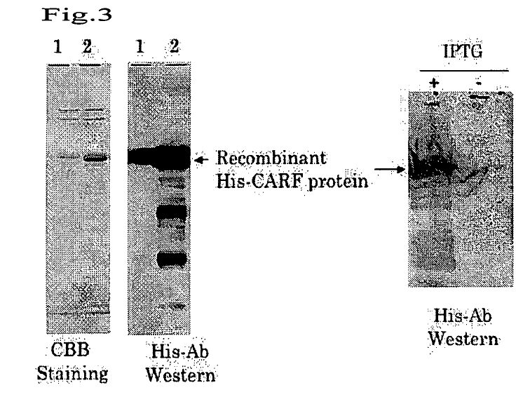

The full-length CARF cDNA (SEQ ID NO: 1) obtained above was incorporated into Sal I and BamH I sites on the vector pQE30 (Qiagen) for expression in Escherichia coli. The vector was then introduced into M15 Escherichia coli strain. Escherichia coli was cultured to OD₅₈₀=0.6 and then treated with isopropyl-1-thio-β-D-galactopyranoside (IPTG) (0.2 mM) at 37° C. for 5 hours for inducing expression. The thus expressed His-labeled recombinant protein was bound to Ni-NTA agarose (Qiagen) so that the protein was isolated. The purity and the size of the thus obtained protein were analyzed by Coomassie brilliant blue (CBB) staining after dodecyl sodium sulfate-polyacrylamide gel electrophoresis (SDS-PAGE). The purity and the size were also confirmed by Western blotting using an antibody against a His label (FIG. 3). In FIG. 3, lane 1 and lane 2 were obtained from a sample in an experiment conducted on another day. The left panel indicates the result of CBB staining. The right panel indicates the result of Western blotting.

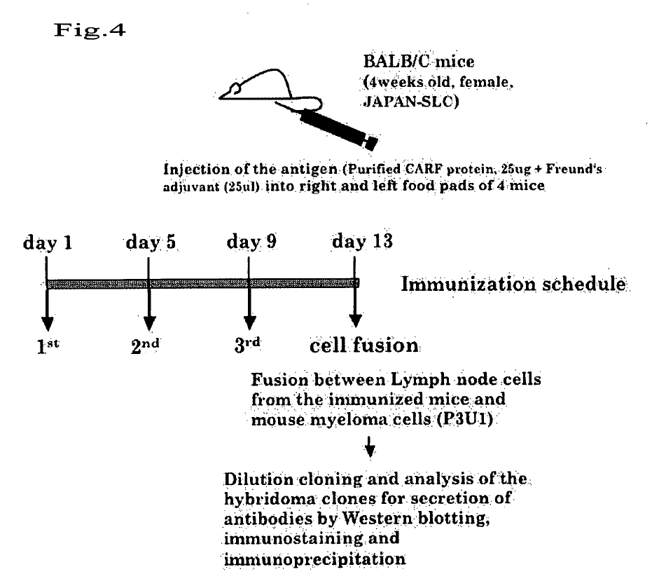

3) Antibody Preparation (See FIG. 4)

The purified CARF protein (SEQ ID NO: 2) obtained in 2) above was mixed with the same amount of Freund's adjuvant. The mixture was used as an antigen for antibody production. For preparation of a monoclonal antibody, the antigen was injected into the footpads of 4-week-old Balb/c mice. The mice were immunized 3 times at intervals of 4 days and then sacrificed on day 13. Lymph node cells obtained from the immunized mice were fused to mouse myeloma cells (P3U1). Hybridoma clones were subjected to dilution cloning. The amount of the antibody secreted in the case of each clone was examined by Western blotting and immunostaining methods.

Meanwhile, for a preparation of a polyclonal antibody, 50 μg of the antigen was injected several instances (twice at intervals of 10 days) into subcutaneous sites on the back of each rabbit. Subsequently, 100 μg of the protein was injected for booster immunization. On day 10 after booster immunization, blood was collected from the rabbits. The serum samples were subjected to Western blotting, immunostaining, and immunoprecipitation analysis for examination of the anti-CARF antibodies.

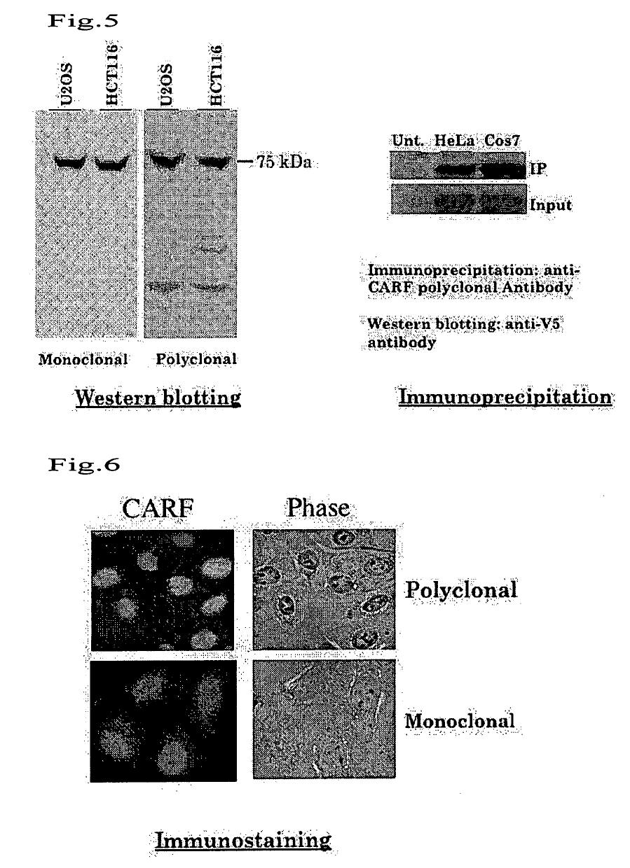

The results of Western blotting are shown on the left in FIG. 5. A cell extract containing 10 μg of the protein was subjected to SDS-PAGE, transferred to a PVDF membrane, and then detected using the anti-CARF antibodies. Detection was performed using a secondary antibody labeled with horseradish peroxidase (HRP) and an ECL detection kit (Amersham). In the case of the monoclonal antibody, only one band was observed in the vicinity of 75 kDa. Furthermore, in the case of the polyclonal antibody, almost only one band was observed in the vicinity of 75 kDa.

The results of the immunoprecipitation method are shown on the right in FIG. 5. The V5-tagged CARF protein was expressed in cells and then immunoprecipitated (IP) using the anti-CARF antibodies. The precipitated protein was western blotted using the anti-V5 tag antibody, so that V5-tagged CARF was detected. The only one protein was also detected in this case. “Input lane” indicates the V5-tagged CARF protein in HeLa cells and Cos7 cells. These results show that the anti-CARF antibodies are specific to CARF antibodies.

Furthermore, FIG. 6 shows the result of immunostaining of the CARF protein using the anti-CARF antibody. Stained nuclei were observed in the case of the anti-CARF monoclonal antibody and in the case of the anti-CARF polyclonal antibody. This was equivalent to the localization of the anti-V5 antibody when the V5-tagged CARF protein had been expressed in cells. It was demonstrated by these results that both anti-CARF antibodies are specific to CARF and inactivate the CARF protein.

1. An anticancer agent which comprises an agent for suppressing CARF expression or an agent for inactivating CARF as an active ingredient.

2. The anticancer agent according to claim 1, wherein the anticancer agent is an agent for inducing senescence or apoptosis of cancer cell.

3. The anticancer agent according to claim 1, wherein the agent for suppressing CARF expression is siRNA or siRNA expression vector to target CARF.

4. The anticancer agent according to claim 3, wherein the siRNA is a double-stranded oligonucleotide consisting of the sense strand represented by SEQ ID NO: 5 and the antisense strand represented by SEQ ID NO: 6.

5. The anticancer agent according to claim 1, wherein the agent for inactivating CARF is an antibody against CARF.

6. The anticancer agent according to claim 5, wherein the antibody against CARF is a monoclonal antibody.


SEQUENCE LISTING (8 sequences)

SEQ ID NO: 1 - DNA, 2682 bp, Homo sapiens
  CDS (152)..(1891)
cggcggctctgggcgctgttgtttggtctttaggcctgcggaggggcgttatctggagagccgcgggtgcaggccgcagtgacagggccgctcgccccgctagtcctgcctgtctcccggtgcagctgtgttcgcggcctgcaggcccaacatggcgcaggaggtgtcggagtacctgagccagaacccgcgggtggcagcctgggtggaggcgctgcgctgcgacggcgagactgacaaacactggcgccaccgccgggattttttgcttcgcaacgccggggacctggcccccgctggcggcgctgcctccgctagcacggatgaagctgccgacgccgagagcgggacccgaaaccggcagctgcagcagctcatctccttttccatggcctgggcgaaccacgtcttcctcgggtgccgataccctcaaaaagttatggataaaatacttagtatggctgaaggcatcaaagtgacagatgctccaacctatacaacaagagatgaactggttgccaaggtgaagaaaagagggatatcgagtagcaatgaaggggtagaagagccatccaaaaaacgagttatagaaggaaaaaacagttctgcagttgagcaagatcacgcaaaaacctctgccaagacagaacgtgcatcagctcagcaggaaaacagttcaacgtgtatagggtcggccatcaaatcagagagtgggaactcagctcggagctctggcatctccagtcagaatagctctacaagtgatggagatcgatctgtttccagccaaagcagcagcagcgtttcctctcaggtaacaacggcaggatctgggaaagcttctgaagcagaagctccagataaacacggttctgcatcatttgtttccttgctgaaatccagtgtgaatagtcacatgacccaatccactgattctagacaacaaagtggatcacctaaaaagagtgctttggaaggctcttcagcctcagcttctcaaagcagctcagagatcgaggtgcccttgttgggctcctcaggaagctcagaggtagaattgccactattgtcttccaaacctagttcagagacagcttcaagtgggttaacttccaaaactagttcagaggcaagtgtttcatcatcagttgctaaaaacagttcctcatcaggcacatccttactgactcccaagagcagctcttcaacaaatacatcgctgctaacttccaagagcacttcccaggtagctgcatcactactagcttccaagagcagctcccagaccagtggatctctggtttccaaaagcacttccttagcaagtgtgtcccagttggcttctaagagtagttctcagactagcacctcacagttgccttctaaaagtacttcacagtcaagtgagagttctgtcaaattctcttgcaagttaaccaatgaagatgtgaaacagaagcaaccttttttcaatagactatataaaacggtggcatggaagttggtagctgttggtggctttagtcccaatgtgaatcatggagagctcctaaatgcagctattgaggctctgaaagcaacactggatgtattttttgtcccactaaaagaattggcagatctgcctcaaaataagagctctcaagaaagtattgtttgtgaattgaggtgcaagtctgtgtatttgggcactggctgtggaaaaagcaaagaaaatgcaaaagcagttgcatcaagagaagcattgaagttatttctcaagaaaaaggtggtggtaaaaatatgtaaaaggaaatacagaggcagtgaaatagaagatctagtactccttgatgaagaatcgaggcctgtaaacttacctccagcactaaaacatcctcaagaattactataatgtgtccaaaatatcactgcatacaatatctggtatttgaagagaaaaactgacttttgtatagtataaaacacaggctttcacaaattttgtattgctttttttccagttttgcagaaaatttacattctagttctcttcacacagtagcagttgtaaataatttatgaatgacagtacacattaaaaggtatgcattagcagcatattagtatgctgttttatttgctgaagaaaatactgtcttctatttttaatgatacattaggtacgatgtgtagttcggtagagtcctaaaatttttgtactactttcaatttggtgaaaatgtattaagttgtctaccatgttttcttttctagctgaataaaccacatcaaaggaaagggaccacagtatttgaatgtttgaaagtctgtaaagcttaaggttttaaaaatgttgcccgtaatgttgaacgtgtctgttaaaaaataaaagaaaaaatagttgcttcaaactatttttatgagaagttgtaagcattttttagatataaagcagtataaagtacttgttattttactctgaagttgtttaaaattcaccatgactttgaccgctgaagattctttaagcgggttaatttatgttttgaggtggaatacaatttacacttttttcttaaaaacatgaatgtgggtttctatattaagcatattttgtgactactattaacagattgatttgtttagatattaaatgctttaagctattttaccttttcaaaaaaaaaaaaaaaaaaaaaa

SEQ ID NO: 2 - protein, 580 aa, Homo sapiens
MAQEVSEYLSQNPRVAAWVEALRCDGETDKHWRHRRDFLLRNAGDLAPAGGAASASTDEAADAESGTRNRQLQQLISFSMAWANHVFLGCRYPQKVMDKILSMAEGIKVTDAPTYTTRDELVAKVKKRGISSSNEGVEEPSKKRVIEGKNSSAVEQDHAKTSAKTERASAQQENSSTCIGSAIKSESGNSARSSGISSQNSSTSDGDRSVSSQSSSSVSSQVTTAGSGKASEAEAPDKHGSASFVSLLKSSVNSHMTQSTDSRQQSGSPKKSALEGSSASASQSSSEIEVPLLGSSGSSEVELPLLSSKPSSETASSGLTSKTSSEASVSSSVAKNSSSSGTSLLTPKSSSSTNTSLLTSKSTSQVAASLLASKSSSQTSGSLVSKSTSLASVSQLASKSSSQTSTSQLPSKSTSQSSESSVKFSCKLTNEDVKQKQPFFNRLYKTVAWKLVAVGGFSPNVNHGELLNAAIEALKATLDVFFVPLKELADLPQNKSSQESIVCELRCKSVYLGTGCGKSKENAKAVASREALKLFLKKKVVVKICKRKYRGSEIEDLVLLDEESRPVNLPPALKHPQELL

SEQ ID NO: 3 - DNA, 21 bp, Artificial Sequence
  Description of Combined DNA/RNA Molecule Synthetic siRNA
  Description of Artificial Sequence Synthetic siRNA
aagaccgaguccaugaggcut

SEQ ID NO: 4 - DNA, 21 bp, Artificial Sequence
  Description of Combined DNA/RNA Molecule Synthetic siRNA
  Description of Artificial Sequence Synthetic siRNA
gccucauggacucggucuuut

SEQ ID NO: 5 - DNA, 21 bp, Artificial Sequence
  Description of Combined DNA/RNA Molecule Synthetic siRNA
  Description of Artificial Sequence Synthetic siRNA
cggaguaccugagccagaaut

SEQ ID NO: 6 - DNA, 21 bp, Artificial Sequence
  Description of Combined DNA/RNA Molecule Synthetic siRNA
  Description of Artificial Sequence Synthetic siRNA
uucuggcucagguacuccgut

SEQ ID NO: 7 - DNA, 21 bp, Artificial Sequence
  Description of Artificial Sequence Synthetic Primer
ggatccatggcgcaggaggtg

SEQ ID NO: 8 - DNA, 22 bp, Artificial Sequence
  Description of Artificial Sequence Synthetic Primer
gtcgactagtaattcttgagga